(19) United States
(12) Patent Application Publication
(10) Pub. No.: US 2016/0008145 A1
(43) Pub. Date: Jan. 14, 2016

(54) Breast Prosthesis Form

(71) Applicant: Christina Riad, McComb, MS (US)

(72) Inventor: Christina Riad, McComb, MS (US)

(21) Appl. No.: 14/863,515

(22) Filed: Sep. 24, 2015

Related U.S. Application Data
(63) Continuation-in-part of application No. 13/373,346, filed on Nov. 12, 2011.

(51) Int. Cl. A61F 2/52

(52) U.S. Cl. CPC A61F 2/52

(57) ABSTRACT

A prosthetic breast form and a method of making thereof provides for a flexible stretchable substantially round or crescent-shaped pillow-like soft pouch adapted to be filled with a desired quantity of granular material to simulate shape and volume of a natural human breast. The pouch has an opening formed in a seam and a pair of closure members is fitted inside the opening. When the closure members are closed they form a visual continuation of the seam of the pouch. The method provides for the user to fill the pouch with the desired quantity of the filler material suitable for a particular lingerie item or garment. In the preferred embodiment, the granular filler comprises waterproof non-spherical beads.

### CROSS-REFERENCE TO RELATED APPLICATIONS

This application is a continuation-in-part of my co-pending application Ser. No. 13/373,346 filed on Nov. 12, 2011 entitled “A Breast Prosthesis Form,” the full disclosure of which is incorporated by reference herein and priority of which is hereby claimed.

### BACKGROUND OF THE INVENTION

Breast cancer often results in mastectomy (removal of the breast). One of the options following the procedure is reconstructive surgery; another option is to use breast prosthesis. Even when choosing the reconstructive surgery, patients have to wait more than six months before the breast reconstruction can be made, allowing the tissue sufficient time to heal. In such cases, mastectomy products, such as external breast forms are needed. Prosthesis offers various advantages, such as protection of the patient's chest and surgical scars; prosthesis helps balance the posture and keeps the bra from shifting side to side or riding up. Importantly, prosthesis helps prevent problems with curvature of the spine, shoulder drop, and muscular pain in the neck and back.

Prosthetic forms are usually positioned in post mastectomy swimsuits or post mastectomy bras. The basic requirements for mastectomy lingerie such as mastectomy bras, mastectomy sleepwear or mastectomy swimsuits is conformance to the natural feel and comfort, as well as size adjustment for different types of uses. It is recognized that the prosthetic forms can differ depending on the type of garment worn by the user.

Conventionally, there exist several types of external breast prosthesis. One type of prosthesis is made of silicone and designed to model natural breast tissue. The prosthesis is designed to weigh the same as the natural breast to help prevent shoulder drop and poor balance. Nonsilicone breast prosthesis is a lightweight breast form made of foam or fiberfill and can be worn right after a mastectomy. It can also be worn during exercise, swimming, and in hot weather.

Still another type is made as an attachable breast, which is fastened to the chest wall using adhesive strips. There also exists a postsurgical soft-form camisole, which is made of a soft, stretchy material with lace elastic straps and is often worn immediately after a mastectomy, lumpectomy (removal of the tumor and some tissue around the tumor), radiation therapy to the chest, or reconstructive breast surgery. A lightweight, removable soft breast form can be placed into a pocket in the camisole. Partial breast prosthesis, also called an equalizer or enhancer, is made of foam, fiberfill, or silicone. It is designed to be worn over a woman's own breast tissue to create a fuller appearance after a part of her breast is removed, such as with a lumpectomy.

Current prosthetic choices require a physician's prescription, are heavy, hot to the skin and rather expensive, ranging from $400 to $600. Additionally, the conventional prosthetic forms require a special brassiere with a pouch to accommodate the prosthesis. Moreover, the conventional prosthetic forms offer no adjustability on terms of weight or shape of the form. It is recognized that the weight of the post mastectomy prosthesis or post mastectomy breast form is critical for comfort. When pockets are sewn in post-mastectomy lingerie, it is important that the prosthesis is kept in proper position during activities. Fashion is of utmost importance when post mastectomy swimsuits, post mastectomy bras, post mastectomy sleepwear and post mastectomy lingerie is designed.

However, conventional prosthetic forms do not allow the user to adjust the shape or size of the prosthesis depending on the type of lingerie or garment being worn by the user. As a result, the patients have to buy several types and shapes of prosthetic forms, which can be expensive. The present invention contemplates elimination of drawbacks associated with conventional prosthetic forms and provision of a prosthetic form that the user can modify by size and weight.

### SUMMARY OF THE INVENTION

It is, therefore, an object of the present invention to provide a breast prosthetic form that a user can modify depending on the desired size and weight.

It is another object of the invention to provide a kit for making a breast prosthetic form.

It is a further object of the invention to provide a kit for making breast prosthetic forms that easily adjusts to the user's garment.

It still a further object of the invention to provide a prosthetic breast form that has a waterproof filler made of irregularly shaped and/or non-spherical beads.

These and other objects of the invention are achieved through a provision of a kit for making a prosthetic breast form to be worn external to a human body that allows a user to make the prosthetic form depending on the desired color, weight, or size of the prosthesis. The kit has at least one, and preferably a plurality of soft flexible stretchable substantially round pillow-like pouches having a closeable seam opening and a pre-determined quantity of granular filler adapted to simulate weight and volume characteristics of a human breast.

An alternative embodiment comprises a crescent-shaped prosthetic breast form having two arcuate surfaces with an opening in one of the arcs for depositing the filler beads into the pouch. A funnel is provided to facilitate depositing of the desired quantity of the granular filler into the pouches.

### BRIEF DESCRIPTION OF THE DRAWINGS

Reference will now be made to the drawings, wherein like parts are designated by like numerals, and wherein

FIG. 1 is a perspective view of the breast prosthetic form according to the present invention.

FIG. 2 is a cross-sectional view of the breast prosthetic form illustrating a step of depositing the filler material into a pouch.

FIG. 3 is a cross sectional view of the prosthetic form filled with the granular filler material and with the seam opening closed.

FIG. 4 illustrates position of the breast prosthetic form of the present invention in a bra.

FIG. 5 illustrates another embodiment of the prosthetic breast form according to the present invention.

### DETAIL DESCRIPTION OF THE INVENTION

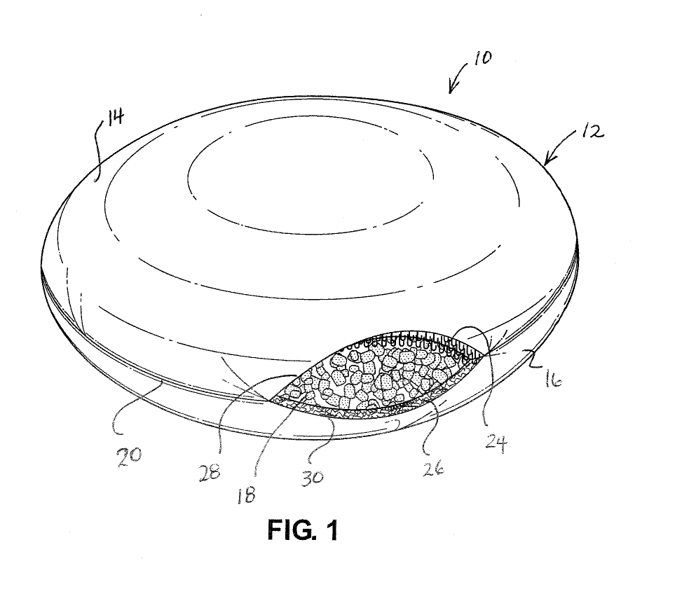

Turning now to the drawings in more detail, FIG. 1 depicts a breast prosthetic form 10 according to the present invention. The prosthetic breast form 10 can be shaped as a round or pillow-like breast form. The prosthetic breast form 10 comprises a pouch 12 formed with a first pouch member 14 and a second pouch member 16, which is a mirror image of the first pouch member 14. The pouch 12 can be fabricated from a soft, flexible, somewhat stretchable material, such as for instance polyester/nylon blend fabric or nylon/spandex fabric, etc. In one exemplary embodiment, the pouch fabric is a sheer stretch mesh nylon/spandex fabric, which contains 80% nylon and 20% spandex. Alternatively, the pouch can be made of a composition of nylon and a mixture of spandex, which provides the elasticity and form-fitting that is required for the purposes of this invention.

Variations in the materials used for manufacturing the pouch 12 include microfibers blended with nylon, silk, cotton, or wool. In one aspect of the invention, the pouch 12 is formed from a blend of nylon net material similar to the material of pantyhose. The pouch 12 can be composed of double knit fabric consisting of elastic polyurethane fiber, such as Lycra, and nylon, or cotton and elastic polyurethane fiber, such as a Lycra material. The pouch 12 can be formed from a material selected from the group consisting of nylon net, spandex, polyester, and microfibers blended with polyester, nylon and spandex.

The pouch 12 can be formed in various sizes, and it is envisioned that a kit for making the breast forms 10 will contain various size and color pouches. The kit contains at least one and in the preferred embodiments, a plurality of pouches 12. It is envisioned that the pouches 12 can be reused by the customer or user.

Each pouch 12 is formed as a substantially round pillow-like pouch formed from two planar-shaped round pouch members 14 and 16. The circumferential edges of the pouch members 14 and 16 are stitched together about almost entire circumference except a small area where a filler opening 18 is formed. It will be understood that the term “substantially round” refers to the general shape of the pouch and that small deviations from a perfectly round shape are possible during manufacturing process when the edges of the pouch members 14 and 16 are stitched together.

When secured together, the circumferential edges of the pouch member 14 and 16 form a continuous, thin and flexible circumferential seam 20, so that the outer boundary of the pouch 12 is somewhat stretchable and deformable allowing the pouch shape to readily change to conform to the shape of the prosthesis, as will be described in more detail hereinafter.

A closure member 22 is secured in the side opening 18. The closure member 22 is formed from two matching closure members or closure strips 24 and 26, which are secured along an open edge 28, 30 of the pouch members 14 and 16, respectively. In one exemplary embodiment, the closure members 24, 26 are formed from small narrow strips of hook-and-loop fasteners, for instance Velcro®. When closed, the closure members 24, 26 are positioned inside the pouch, forming a visual continuation of the circumferential seam 20 so that the seam 20 appears continuous.

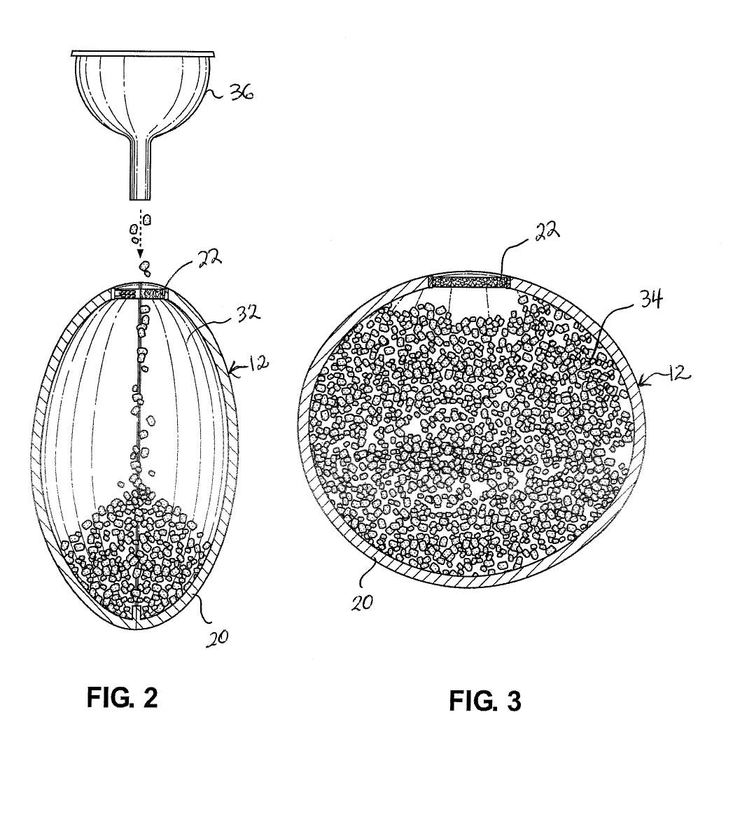

An empty pouch 12 defines an inner chamber 32 configured to receive a filler material 34 therein. The pouch 12 can collapse, as illustrated in FIG. 2, and assume an oval shape as it is being filled with the filler material 34. As the pouch 12 is filled with the filler material 34 the pouch 12 gradually unfolds and obtains the desired round pouch shape.

A pre-determined quantity of the granular filler material 34 is provided in the kit for depositing into the pouch 12 and forming the prosthetic form 10. The granular filler material 32 can be made from a variety of free-flowing granules, including irregularly shaped and elongated granules. A preliminary research indicated that round beads tend to role and swish, which leads to an uncomfortable feel and look. Therefore, the filler 34 of the present invention preferably contains non-spherical beads.

In one of the preferred embodiments, the granules of the granular filler 34 are selected to be between 0.5 mm (0.0197 inch) in length, although it is envisioned that in some cases, granules of up to 7 mm (0.276 inch) can be used as well. If elongated granules are used, it is envisioned that the length/width ratio of the granules would not exceed 3. In empirical tests, the filler material was selected to have 1 cup (0.237 liter) of the filler material equal to about 240 cc. In one of the exemplary embodiments, the filler material 32 has lentil-shaped granules or beads.

In the preferred embodiment, the combined weight and volume of the filler simulates the human breast tissue. The granular filler material 34 can be made of waterproof inorganic matter, natural or artificial, such as for instance polystyrene, micro beads, micro polystyrene, polypropylene pellets, sodium aluminosilicate, acrylonitrile butadiene styrene (ABS), etc.

In a kit according to the present invention, the amount of the filler material corresponds to the selected size of the pouch as well as the number of the pouches that can be purchased. The type of the filler material 34 can be a mixture of lightweight and heavy weight beads to allow the customer to adjust the desired weight and volume of the prosthesis.

The kit 10 further comprises a channeling tool, such as a funnel 36 for pouring the fine-grained substance of the filler material 34 into the pouch 12. The funnel 36 can be made from stainless steel, aluminum, plastic, or other suitable non-reactive non-corrosive materials. The kit according to the present invention can come in a box (not shown), wherein one or more pouches 12, the granular filler 34, and the funnel 36 are packaged.

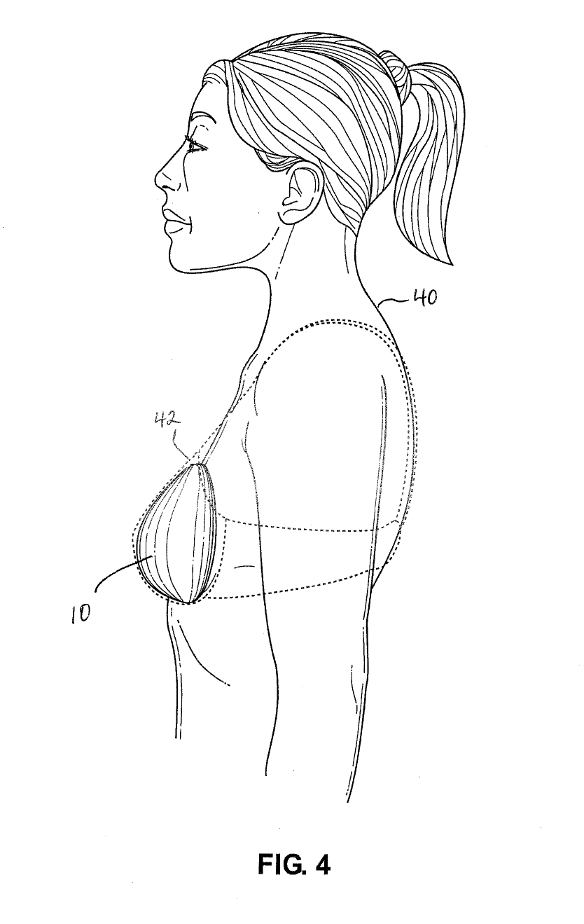

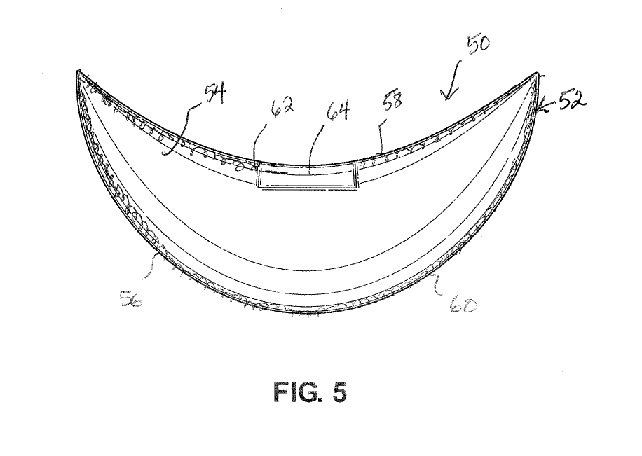

Turning now to the second embodiment of the invention illustrated in FIG. 5, the prosthetic breast form 50 is shown having a substantially crescent configuration. The crescent-shaped form 50 comprises a pillow-like pouch 52 formed from two planar members (only one planar member 54 is seen in FIG. 5) that are secured together along their peripheral edges by a seam 56, such as for instance by stitching. It will be understood that the term “substantially crescent” refers to the general shape of the pouch and that small deviations from a perfectly crescent shape are possible during manufacturing process when the edges of the pouch members are stitched together. Similarly to the embodiment of FIGS. 1-4, the embodiment of FIG. 5 has two pouch members configured as mirror images of each other.

In the illustration of FIG. 5, the pouch 52 has a first arcuate surface 58 and a second arcuate surface 60 opposite the first arcuate surface 58. The radii of the arcs formed by the first arcuate surface 58 and the second arcuate surface 60 are not equal. It is envisioned that the first arcuate surface 58 can be formed as extending along a straight line in cross-section, which configuration can be obtained when a round form is cut in half along a diametric line. Both shapes, half-circle and two-arc crescent, are acceptable embodiments and understanding of “substantially crescent” within the teaching of the instant invention. In the half-circle (half-moon) embodiment, one surface of the pouch 52 will be arcuate, and another—planar, straight in cross-section. Both such “crescent configuration” embodiments have at least one arcuate surface.

A closure opening 62 is formed in the seam 56 in the first surface 58. A pair of mating hook-and-loop fastener strips or closure members 64 is secured within the opening 62.When secured together, the fastener strips 64 are positioned inside an inner chamber defined by the pouch 52. The fastener strips form a visual continuation of the peripheral seam when the closure opening 62 is closed The closure opening 62 allows access to the inner chamber for depositing the granular filler 34 therein. The material for making the pouch 52 is similar to the material for forming the pouch 12.

In operation, the user 40 selects one of the pouches 12, 52 depending on the size of the breast form the user wishes to make. The user then positions the funnel 36 into the opening 18, 62 of the selected pouch and deposits the granular filler 34 through the funnel 36 into the pouch 12, 52 as illustrated in the example of FIG. 2. The user 40 then presses the hook-and-loop fastener strips 24, 26 (64), thereby closing the pouch and preventing the filler granules from escaping from the pouch 12, 52. The user then positions the filled pouch 12 or 52 which becomes the prosthetic breast form 10, 50 in a cup of a bra 42, of a swimsuit or a pocket of a mastectomy brassiere. Since the pouch is flexible, stretchable and elastic, the prosthetic breast form 10, 50 immediately conforms to the body of the user 40 and to the cup 42 of the bra worn by the user. If the user changes the garment or the lingerie, the user can use other pouch bodies for making different size forms. Alternatively, the user can open the pouch 12, 52 add more granular material or remove some of the granular filler 34 to accommodate the desired size and mass. These adjustments can be done in the privacy of the user's home without the need for special assistant or technician. If a strong net material is selected for the pouches 12, 52 the pouches can be reused numerous times.

The prosthetic breast forms 10, 50 comfortably adjust to the woman's chest wall and to the mastectomy bra. The prosthetic breast forms 10, 50 readily shape to the contours of the chest wall formed after a surgery. It is envisioned that the granular material and mass thereof can be varied in order to provide the size, shape and weight suitable for matching the woman's natural breast. It is envisioned that the breast forms can be used to produce a breast enhancement appearance by users not necessarily following a mastectomy procedure.

The breast forms 10, 50 can be purchased as a single or pair. Since the beads are waterproof the breast forms 10, 50 can be used during swimming and other types of exercises. It is envisioned that the pouches 12, 52 can be provided in different sizes, from extra small to extra large. For instance, an extra small pouch can be 4 inches in diameter, and an extra-large pouch can be 7 inches in diameter.

Many changes and modifications can be made in the design of the present invention without departing from the spirit thereof. I, therefore, pray that my rights to the present invention be limited only by the scope of the appended claims.

I claim:

1. A kit for making a prosthetic breast form to be worn external to a human body, comprising:
at least one substantially round pillow-like flexible stretchable pouch having a circumferential seam and a closure member fitted in the seam;
a pre-determined quantity of granular filler adapted to simulate weight and volume characteristics of a human breast; and
a funnel means for depositing the granular filler into said at least one pouch.

2. The kit of claim 1, wherein said granular filler comprises a waterproof granulated material with granules of between 0.5 mm and 7 mm.

3. The kit of claim 2, wherein the granules are non-spherical beads.

4. The kit if claim 2, wherein the granules are elongated beads having length to width ratio of less than 3.

5. The kit of claim 1, wherein the granular filler is made from a material selected from a group consisting of natural and artificial materials.

6. The kit of claim 1, wherein the pouch comprises a pair of mirror-image circular pouch members, each pouch member having a circumferential edge.

7. The kit of claim 6, wherein circumferential edges of the pouch members are secured together to form a circumferential seam with a side opening.

8. The kit of claim 7, wherein the closure member comprises a pair of hook-and-loop fastener strips secured in mating relationship in the side opening inside the pouch.

9. The kit of claim 7, wherein the fastener strips form a visual continuation of the circumferential seam when the side opening is closed.

10. The kit of claim 1, wherein said at least one pouch is formed from a material selected from the group consisting of nylon net, spandex, polyester, and microfibers blended with polyester, nylon and spandex.

11. A prosthetic breast form configured to be worn external to a human body, the form comprising a substantially round pillow-like flexible stretchable pouch having a circumferential seam and a closure member fitted in the seam, said pouch defining an inner chamber, and a predetermined quantity of granular filler deposited in the inner chamber, said filler material being adapted to simulate physical characteristics of a natural human breast.

12. The prosthetic breast form of claim 11, wherein said granular filler comprises a waterproof granulated material with granules of between 0.5 mm and 7 mm.

13. The prosthetic breast form of claim 12, wherein the granules are non-spherical beads.

14. The prosthetic breast form of claim 12, wherein the granules are elongated beads having length to width ratio of less than 3.

15. The prosthetic breast form of claim 11, wherein the granular filler is made from a material selected from a group consisting of natural and artificial materials.

16. The prosthetic breast form of claim 11, wherein the pouch comprises a pair of mirror-image circular pouch members, each pouch member having a circumferential edge.

17. The prosthetic breast form of claim 16, wherein circumferential edges of the pouch members are secured together to form a circumferential seam with a side opening.

18. The prosthetic breast form of claim 17, wherein the closure member comprises a pair of hook-and-loop fastener strips secured in mating relationship in the side opening inside the pouch.

19. The prosthetic breast form of claim 17, wherein the fastener strips form a visual continuation of the circumferential seam when the side opening is closed.

20. The prosthetic breast form of claim 11, wherein said at least one pouch is formed from a material selected from the group consisting of nylon net, spandex, polyester, and microfibers blended with polyester, nylon and spandex.

21. A method of manufacturing a prosthetic breast form to be worn external to a human body, comprising the steps:
providing at least one substantially round pillow-like flexible stretchable pouch having a circumferential seam and a closure member fitted in the seam;
providing a pre-determined quantity of granular filler adapted to simulate weight and volume characteristics of a human breast;
depositing a sufficient quantity of the granular filler into the at least one pouch to achieve a desired size of the prosthetic form to simulate a natural human breast; and
closing the closure member after the granular filler is deposited into the at least one pouch.

22. The method of claim 21, further comprising a step of providing a funnel to facilitate deposit of the granular filler into the at least one pouch.

23. The method of claim 21, wherein said at least one pouch is formed from a material selected from the group consisting of nylon net, spandex, polyester, and microfibers blended with polyester, nylon and spandex.

24. The method of claim 21, wherein said granular filler comprises a waterproof granulated material with granules of between 0.5 mm and 7 mm.

25. The method of claim 21, wherein the granular filler comprises non-spherical beads.

26. The method of claim 21, wherein the granular filler comprises elongated beads having length to width ratio of less than 3.

27. The method of claim 21, wherein the at least one pouch comprises a pair of mirror-image circular pouch members, each pouch member having a circumferential edge.

28. The method of claim 27, wherein circumferential edges of the pouch members are secured together to form a circumferential seam with a side opening.

29. The method of claim 28, wherein the closure member comprises a pair of hook-and-loop fastener strips secured in mating relationship in the side opening inside the pouch.

30. The method of claim 28, wherein the fastener strips form a visual continuation of the circumferential seam when the side opening is closed.

31. A prosthetic breast form configured to be worn external to a human body, the form comprising a substantially crescent pillow-like flexible stretchable pouch having at least one arcuate surface, a peripheral seam, and a closure member fitted in the seam, said pouch defining an inner chamber, and a predetermined quantity of granular filler deposited in the inner chamber, said filler material being adapted to simulate physical characteristics of a natural human breast.

32. The prosthetic breast form of claim 31, wherein said granular filler comprises a waterproof granulated material with granules of between 0.5 mm and 7 mm.

33. The prosthetic breast form of claim 32, wherein the granules are non-spherical beads.

34. The prosthetic breast form of claim 32, wherein the granules are elongated beads having length to width ratio of less than 3.

35. The prosthetic breast form of claim 31, wherein the granular filler is made from a material selected from a group consisting of natural and artificial materials.

36. The prosthetic breast form of claim 31, wherein the pouch comprises a pair of mirror-image crescent-shaped pouch members, each pouch member having a peripheral edge.

37. The prosthetic breast form of claim 36, wherein peripheral edges of the pouch members are secured together to form a peripheral seam with a closure opening.

38. The prosthetic breast form of claim 37, wherein the closure member comprises a pair of hook-and-loop fastener strips secured in mating relationship in the closure opening inside the pouch.

39. The prosthetic breast form of claim 37, wherein the fastener strips form a visual continuation of the peripheral seam when the closure opening is closed.

40. The prosthetic breast form of claim 31, wherein said at least one pouch is formed from a material selected from the group consisting of nylon net, spandex, polyester, and microfibers blended with polyester, nylon and spandex.

41. The prosthetic form of claim 31, wherein the crescent pouch has a first arcuate surface and a second arcuate surface opposite the first arcuate surface.

42. The prosthetic form of claim 41, wherein the first arcuate surface has unequal radius with a radius of the second arcuate surface.
